(19) United States
(12) Patent Application Publication
(10) Pub. No.: US 2026/0306076 A1
(43) Pub. Date: Oct. 8, 2026

(54) GUT-SAMPLING CAPSULE WITH LATERAL OPENING AND MULTIPLE SAMPLING COMPARTMENTS

(71) Applicant: TRUSTEES OF TUFTS COLLEGE, Medford, MA (US)

(72) Inventors: Sameer SONKUSALE, Lincoln, MA (US); Ruben DEL RIO RUIZ, Candia, NH (US)

(21) Appl. No.: 19/482,393

(22) PCT Filed: May 7, 2024
(86) PCT No.: PCT/US2024/028135

§ 371 Date: Nov. 7, 2025

Related U.S. Application Data
(60) Provisional application No. 63/464,679, filed on May 8, 2023.

(51) Int. Cl. A61B 10/00; A61B 5/00

(52) U.S. Cl. CPC A61B 10/0045; A61B 5/7285; A61B 2010/0061; A61B 2562/162

(57) ABSTRACT

A capsule for traversing a gut includes a motor, an inlet, and recesses for receiving a sample from outside said capsule. The motor is configured to cause each of the recesses to enter a sampling position. A recess in a sampling position is aligned with the inlet to permit matter from outside the capsule to be collected within the recess.

### RELATED APPLICATIONS

This application claims the benefit of the May 8, 2023 priority date of U.S. Application 63/464,679, the content of which is incorporated herein by reference.

### STATEMENT OF GOVERNMENT RIGHTS

This invention was made with government support under grant DK132314 awarded by the National Institutes of Health. The government has certain rights in the invention.

### FIELD OF INVENTION

The invention relates to probes for sampling matter from inaccessible locations and in particular to probes for sampling matter from a digestive system.

### BACKGROUND

It is often useful to sample matter from one or more different locations of a human digestive system. Such information is useful for ascertaining the functioning of the digestive system. It is also useful for observations of the gut microbiome.

It is known to sample matter that has been naturally expelled from the digestive system. However, such matter does not provide any spatial selectivity.

### SUMMARY

In one aspect, the invention features a capsule for traversing a gut, the capsule comprising a motor, an inlet, and recesses for receiving a sample from outside the capsule. The motor is configured to cause a first recess one of the recesses to enter a sampling position and to later cause a second one of the recesses to enter the sampling position. A recess in the sampling position is aligned with the inlet to permit matter from outside the capsule to be collected within the recess.

In some embodiments, the capsule comprises a cylinder and the recesses are formed in the cylinder.

In other embodiments, the motor is a stepper motor.

Also among the embodiments are those in which the motor is configured to cause each of the first and second recesses to transition into a blocked position in which no further matter from outside the capsule can be collected within the recess and those in which the motor is configured to cause all of the recesses to be in a blocked position in which no further matter from outside the capsule can be collected within the recess.

In some embodiments, the motor is configured to cause each of the first and second recesses to remain in the sampling position for no longer than a predetermined sampling time.

Embodiments include those in which the capsule comprises a cylinder in which the recesses are formed. Among these embodiments are those in which the motor rotates the cylinder. However, in other embodiments, the motor moves the inlet.

Still other embodiments include those in which the inlet is on a wall of the capsule and wherein the motor is configured to rotate the wall.

Embodiments also include those in which the capsule is an autonomous capsule. Such embodiments feature a sensor for sensing an environment of the gut. The motor is configured to operate in response to information provided by the sensor. Among these are embodiments in which the sensor senses hydrogen ion concentration and embodiments in which the sensor senses dissolved oxygen concentration.

Still other embodiments are those in which the motor is configured to be actuated from outside of the capsule. Among these are embodiments in which the motor is configured to be actuated by an externally applied magnetic field. These include embodiments in which the magnetic field interacts with a reed switch within the capsule.

Embodiments further include those that comprise an enteric coating that coats the capsule and dissolves when the capsule has reached a portion of the gut that is to be sampled.

Still other embodiments include a printed-circuit board in the capsule, circuitry mounted on the curved printed-circuit board, and a programming connector coupled to the circuitry. In such embodiments, the circuitry is configured to control the motor. Among these are embodiments in which the printed-circuit board is curved with a radius of curvature that conforms to a wall of the capsule.

Also among the embodiments are those in which the inlet is a lateral inlet and those that further include a cylinder that rotates, wherein each of the recesses spans an angular range along the cylinder's circumferential direction. In such embodiments, there exists an inter-recess surface between each pair of recesses. This inter-recess surface has an extent that is sufficient to completely cover the inlet.

Still other embodiments include those that have more than two recesses.

Also among the embodiments are those in which the capsule includes a power source and a switch that responds to an external magnet. This switch, the presence of a magnetic field, connects the motor to the power source. A suitable switch is a reed switch.

In another aspect, the invention features a method that includes collecting matter from within a gut, wherein collecting the matter comprises providing a capsule to be swallowed. The capsule includes a motor, an inlet, and recesses for receiving a sample from outside the capsule, the recesses comprising at least a first recess and a second recess. The method continues with using the motor to cause the first recess to enter a sampling position, and, after having caused the recess to enter the sampling position, using the motor to cause the second recess to enter the sampling position. Causing a recess to enter the sampling position comprises causing the recess to be aligned with the inlet to permit matter from outside the capsule to be collected within the recess.

These and other features of the invention will be apparent from the following detailed description and the accompanying figures, in which:

### BRIEF DESCRIPTION OF THE DRAWINGS

FIG. 1 is a view of a sampling capsule,

FIG. 2 shows the sampling capsule of FIG. 1 in with its cylinder having been rotated into a sampling position,

FIG. 3 shows a cross-section of the cylinder in the configuration shown in FIGS. 2,

FIG. 4 shows the sampling capsule of FIG. 1 in with its cylinder having been rotated into a blocking position, and

FIG. 5 shows a cross-section of the cylinder in the configuration shown in FIG. 5.

### DETAILED DESCRIPTION

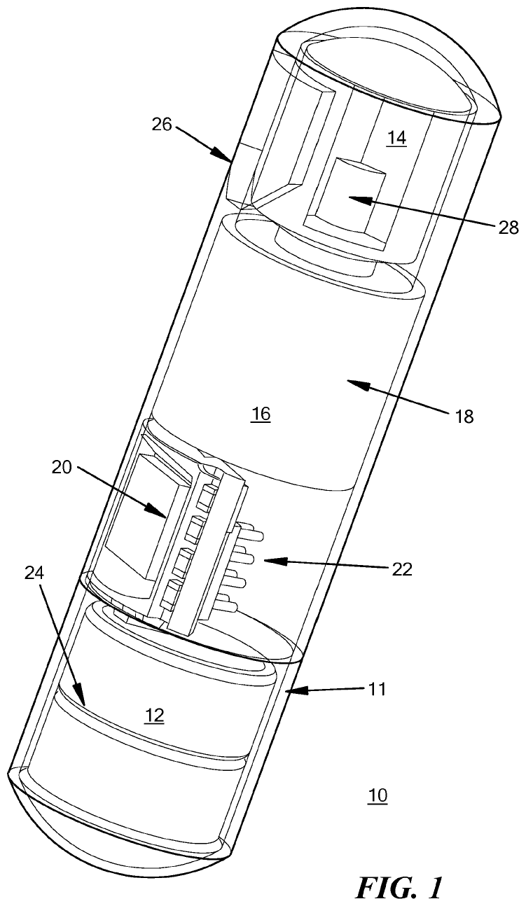

FIG. 1 shows a capsule 10 that is to traverse a gut after having been swallowed. A suitable method for manufacturing such a capsule 10 is to use an additive manufacturing process, such as 3D printing. An enteric coating 11 that coats the capsule 10 dissolves when the capsule has reached a portion of the gut that is to be sampled.

The capsule 10 includes a first compartment 12 and a second compartment 14 at opposite ends thereof. Between the first compartment 12 and the second compartment 14 is a third compartment 16 that includes a motor 18 and circuitry 20 for controlling the motor 18. The circuitry 20 is coupled to a programming connector 22.

In the illustrated embodiment, the circuit elements that comprise the circuitry 20 are disposed on a printed-circuit board within the third compartment 16. However, in other embodiments, the printed-circuit board is curved with a radius of curvature that enables the circuitry 20 to form part of the capsule's wall. This results in either a more compact capsule 10 or a larger volume available for collection of gut matter. Such a circuit board can be manufactured through additive manufacturing as well.

The first compartment 12 contains a power source 24 that provides power for both the circuitry 20 and for the motor 18. A suitable power source 24 is a battery that comprises one or more cells.

The second compartment 14 features a lateral inlet 26 and cylinder 28 that rotates when made to do so by the motor 18. The use of a lateral inlet 26 rather than a distal inlet at the tip of the capsule 10 has been found to promote more efficient collection of a sample.

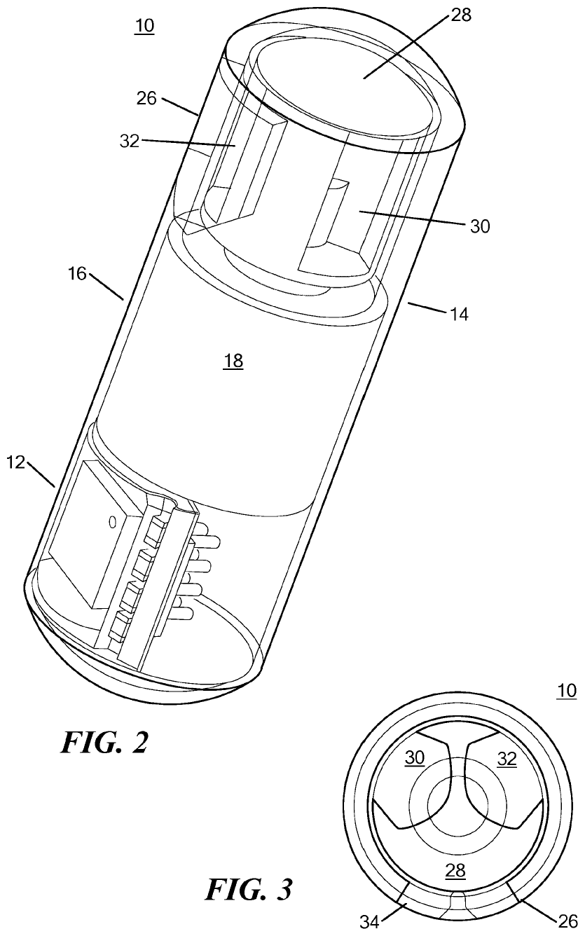

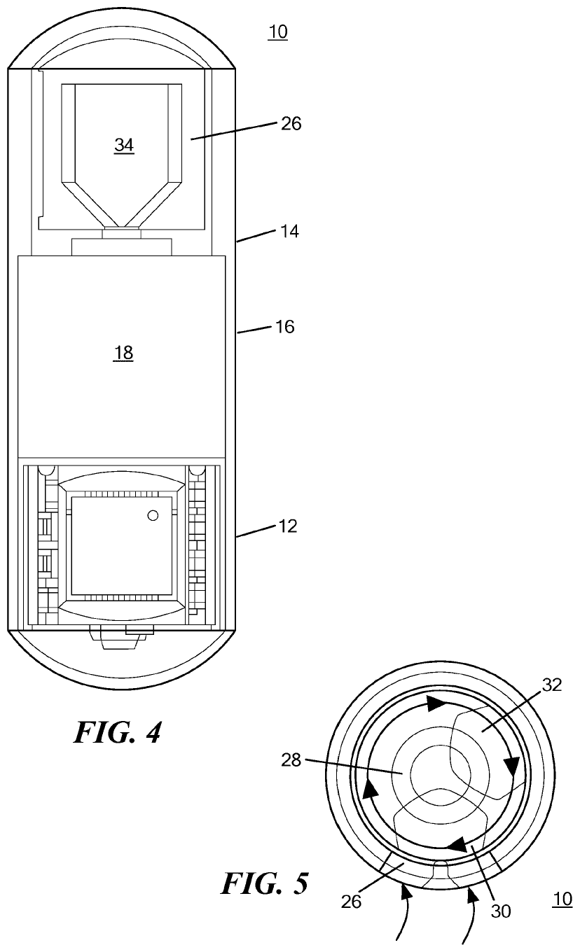

As shown in FIG. 2, the cylinder 28 has recesses 30, 32, each of which spans an angular range along the cylinder's circumferential direction and a longitudinal extent along the cylinder's longitudinal direction. Between each pair of recesses is an inter-recess surface 34 of sufficient extent to completely cover the lateral inlet 26.

The motor 18 rotates the cylinder 28 into either a sampling position or a blocking position. In a preferred embodiment, the motor 18 is a stepper motor. Such an embodiment is particularly advantageous because of its accuracy in positioning the cylinder 28.

To place the cylinder 28 into a sampling position, the motor 18 rotates the cylinder 28 so that one of the recesses 30, 32 faces the inlet 26, as shown in FIGS. 2 and 3. With the cylinder 28 in the sampling position, matter from outside the capsule 10 is able to enter the recess 30, 32 as a result of natural peristaltic pressure.

After a predetermined time, the motor 18 automatically rotates the cylinder 28 into a blocking position, which is shown in FIG. 3. In the blocking position, an inter-recess surface 34 faces the inlet 26. As a result, sample collection ends. At this point, the collected sample is safely sequestered from contamination.

Embodiments of the cylinder 28 include those with two recesses and those with three or more recesses. The number of recesses determines how many locations in the gut can be sampled. However, as the number of recesses increases, the amount of sample that can be collected per location decreases.

It is important to be able to control the motor 18 so that rotation of the cylinder 28 occurs at the correct position in the gut.

In some embodiments, the capsule 10 is autonomous. Such a capsule 10 would include a sensor that detects some property of the gut environment. The property is one that is indicative of the capsule's having reached a particular location. Since the concentration of hydrogen ions varies considerably with location, a suitable sensor is one that senses the concentration of hydrogen ions and triggers the motor 18 accordingly. Since the concentration of dissolved oxygen also varies with location in the gut, another useful sensor is one that senses the concentration of dissolved oxygen.

In other embodiments, the capsule 10 includes a reed switch that responds to an external magnet. In the presence of a suitable magnetic field, the reed switch connects the motor 18 to the power source 24.

In the embodiment described herein, the motor 18 rotates the cylinder. However, only relative motion between the cylinder and the wall is necessary. Thus, other contemplated embodiments include those in which the motor 18 rotates a wall of the second compartment 12 instead of the cylinder 26.

Having described the invention and a preferred embodiment thereof, what is claimed as new and secured by Letters Patent is:

1. An apparatus for collecting matter from within a gut, said apparatus comprising a capsule for traversing a gut, said capsule comprising a motor, an inlet, and recesses for receiving a sample from outside said capsule, said recesses comprising at least a first recess and a second recess, wherein said motor is configured to cause said first recess to enter a sampling position and to later cause said second recess to enter said sampling position, wherein a recess in said sampling position is aligned with said inlet to permit matter from outside said capsule to be collected within said recess.

2. The apparatus of claim 1, wherein said capsule comprises a cylinder and said recesses are formed in said cylinder.

3. The apparatus of claim 1, wherein said capsule comprises a cylinder in which said recesses are formed and wherein said motor rotates said cylinder.

4. The apparatus of claim 1, wherein said inlet is on a wall of said capsule and wherein said motor is configured to rotate said wall.

5. The apparatus of claim 1, wherein said capsule is an autonomous capsule that includes a sensor for sensing an environment of said gut, wherein said motor is configured to operate in response to information provided by said sensor.

6. The apparatus of claim 1, wherein said motor is configured to cause each of said first and second recesses to transition into a blocked position in which no further matter from outside said capsule can be collected within said recess.

7. The apparatus of claim 1, wherein said motor is configured to cause all of said recesses to be in a blocked position in which no further matter from outside said capsule can be collected within said recess.

8. The apparatus of claim 1, wherein said motor is configured to cause each of said first and second recesses to remain in said sampling position for no longer than a predetermined sampling time.

9. The apparatus of claim 1, wherein said motor is configured to be actuated from outside of said capsule.

10. The apparatus of claim 1, wherein said motor is configured to be actuated by an externally applied magnetic field.

11. The apparatus of claim 1, wherein said motor is a stepper motor.

12. The apparatus of claim 1, further comprising an enteric coating that coats said capsule and dissolves when said capsule has reached a portion of said gut that is to be sampled.

13. The apparatus of claim 1, further comprising a curved printed circuit board in said capsule, circuitry mounted on said curved printed-circuit board, and a programming connector coupled to said circuitry, said circuitry being configured to control said motor, wherein said printed-circuit board is curved with a radius of curvature that conforms to a wall of said capsule.

14. The apparatus of claim 1, wherein said inlet is a lateral inlet.

15. The apparatus of claim 1, further comprising a cylinder that rotates, said cylinder defining a circumferential direction and a longitudinal extent, wherein each of said recesses spans an angular range along said circumferential direction, and wherein between each pair of recesses is an inter-recess surface of sufficient extent to completely cover said inlet.

16. The apparatus of claim 1, further comprising at least one additional recess.

17. The apparatus of claim 1, wherein said capsule comprises a sensor for sensing hydrogen ion concentration in said gut, wherein said motor is configured to operate in response to information provided by said sensor.

18. The apparatus of any claim 1, wherein said capsule comprises a sensor for sensing concentration of dissolved oxygen in said gut, wherein said motor is configured to operate in response to information provided by said sensor.

19. The apparatus of claim 1, wherein said capsule comprises a power source and a reed switch that responds to an external magnet, wherein said reed switch, in the presence of a magnetic field, connects said motor to said power source.

20. A method comprising collecting matter from within a gut, wherein collecting said matter comprises providing a capsule to be swallowed, said capsule comprising a motor, an inlet, and recesses for receiving a sample from outside said capsule, said recesses comprising at least a first recess and a second recess, using said motor, causing said first recess to enter a sampling position, after having caused said recess to enter said sampling position, using said motor to cause said second recess to enter said sampling position, wherein causing a recess to enter said sampling position comprises causing said recess to be aligned with said inlet to permit matter from outside said capsule to be collected within said recess.
